(19) United States
(12) Patent Application Publication
(10) Pub. No.: US 2017/0311864 A1
(43) Pub. Date: Nov. 2, 2017

(54) HEALTH CARE ASSISTING DEVICE AND HEALTH CARE ASSISTING METHOD

(71) Applicant: OMRON Corporation, Kyoto-shi (JP)

(72) Inventors: Seiichi MANABE, Kusatsu-shi (JP); Hiromatsu AOKI, Suita-shi (JP)

(73) Assignee: OMRON Corporation, Kyoto-shi (JP)

(21) Appl. No.: 15/653,964

(22) Filed: Jul. 19, 2017

Related U.S. Application Data
(63) Continuation of application No. PCT/JP2015/086238, filed on Dec. 25, 2015.

(30) Foreign Application Priority Data
Feb. 13, 2015 (JP) 2015-026474

(51) Int. Cl. A61B 5/16; G06F 19/00

(52) U.S. Cl. CPC A61B 5/165; G06F 19/34

(57) ABSTRACT

A health care assisting device includes: an image acquisition part that acquires a plurality of images in which a target person is photographed in time series; an expression recognizer that recognizes a feature of an expression of the target person from the plurality of images acquired by the image acquisition part; a storage in which expression recognition results of the plural images are stores as time-series data; a health state estimator that detects a feature associated with a temporal change of the expression of the target person from the time-series data stored in the storage, and estimates a mental health state of the target person based on the detected feature; and an output part that outputs information on the mental health state of the target person based on an estimation result of the health state estimator.

### CROSS-REFERENCE TO RELATED APPLICATION

This application is a continuation application of International Application No. PCT/JP2015/086238, filed on Dec. 25, 2015, which claims priority based on the Article 8 of Patent Cooperation Treaty from prior Japanese Patent Applications No. 2015-026474, filed on Feb. 13, 2015, the entire contents of which are incorporated herein by reference.

### FIELD

The disclosure relates to a technology of assisting mental health care of a person.

### BACKGROUND

Nowadays, the number of patients who suffer from mental diseases such as depression and dementia increases considerably, which becomes a social problem. Particularly, in Japan, it is said that a measure against the mental disease becomes a more critical issue in the future with increasing aged person. It is important to prevent the mental disease in an ordinary lifestyle, and awareness in an early stage (early detection) and proper treatment are a shortcut to recovery. However, few persons have correct recognition and knowledge about the mental disease, and an ordinary person is hardly aware of a sign (indication) or a symptom of the mental disease.

For example, JP 2006-305260 A discloses a device that assists a diagnosis of the mental disease. The device generates a diagnostic data vector in which bilateral symmetry of an expression, tensions of eyes, tension of cheeks, and angles of corners of a mouth are digitized from a facial image of an examinee, and displays graphs of diagnostic results of a degree of paranoia, a degree of neurosis, a degree of sociopath, a degree of depression, and a degree of stress. Indeed, frequently the sign of the mental disease appears as the expression or tension of a face, and a specialist uses a change of a facial expression as one of clues to estimation of a mental health state of the patient in the actual diagnosis or counseling. However, even the specialist can hardly distinguish whether states of the expression, eyes, cheeks, and corners of the mouth are the sign of the mental disease or personality (an original face or a usual expression) only by viewing one facial image. Accordingly, it is considered that high-reliability diagnostic information is hardly obtained by the technique disclosed in JP 2006-305260 A.

### SUMMARY

An object of an embodiment of the present invention is to provide a technology of being able to estimate the mental health state of the person based on the facial expression recognized from the image and provide information useful for the mental health care.

In order to achieve the object, in an embodiment of the present invention, a feature associated with a temporal change of a facial expression of a target person from time-series data of the facial expression is detected, and a mental health state of the target person is estimated based on the detected feature.

Specifically, in accordance with one aspect of the present invention, a health care assisting device is configured to assist mental health care of a target person, the health care assisting device including: an image acquisition part configured to acquire plural images in which the target person is photographed in time series; an expression recognizer configured to recognize a feature of an expression of the target person from the plurality of images acquired by the image acquisition part; a storage in which expression recognition results of the plurality of images are stores as time-series data; a health state estimator configured to detect a feature associated with a temporal change of the expression of the target person from the time-series data stored in the storage, and estimate a mental health state of the target person based on the detected feature; and an output part configured to output information on the mental health state of the target person based on an estimation result of the health state estimator.

Accordingly, the change (improve or worse) of the mental health state that appears as the change of the facial expression can be detected by focusing on the feature associated with the temporal change of the facial expression, and the high-reliability estimation result can be obtained compared with the case that the estimation is performed using only the facial expression in one image. Because the high-reliability estimation result is automatically and early obtained, useful information can properly be provided in response to the mental health state of the target person, and the mental health care of the target person can properly be assisted.

It may be preferable that the health state estimator estimates that the mental health state of the target person becomes worse when detecting a decrease of the expression indicating a positive emotion as the feature associated with the temporal change of the expression. This is because the expression (the expression such as happiness) indicating the positive emotion decreases considerably in a “depressive state” that is one of the signs (indications) or symptoms of mental diseases such as depression and dementia. Alternatively, the health state estimator may estimate that the mental health state of the target person becomes worse when detecting an increase of the expression indicating a negative emotion as the feature associated with the temporal change of the expression. This is because the expression (the expression such as sadness) indicating the negative emotion increases considerably in the “depressive state” that is one of the signs (indications) or symptoms of mental diseases such as depression and dementia.

It may be preferable that the expression recognizer calculates a score in which a degree of each of a plurality of kinds of the expressions is digitized from the image of the target person, and outputs the score of each expression as the expression recognition result, the health state estimator selects or generates a positive expression score indicating the degree of the positive emotion from the scores of the plurality of kinds of the expressions, and the health state estimator estimates that the mental health state of the target person becomes worse when detecting that the positive expression score in a latest predetermined period has a lowering tendency compared with an ordinary value as the feature associated with the temporal change of the expression. Therefore, the lowering of the expression indicating the positive emotion can quantitatively be evaluated, and the worsening of the mental health state can be estimated with high reliability. Alternatively, the health state estimator may select or generate a negative expression score indicating the degree of the negative emotion from the scores of the plurality of kinds of the expressions, and the health state estimator may estimate that the mental health state of the target person becomes worse when detecting that the negative expression score in the latest predetermined period has a rising tendency compared with the ordinary value as the feature associated with the temporal change of the expression. Therefore, the rising of the expression indicating the negative emotion can quantitatively be evaluated, and the worsening of the mental health state can be estimated with high reliability.

It may be preferable that the expression recognizer calculates a score in which a degree of each of a plurality of kinds of the expressions is digitized from the image of the target person, and outputs the score of each expression as the expression recognition result, the health state estimator selects or generates a positive expression score indicating the degree of the positive emotion from the scores of the plurality of kinds of the expressions, and the health state estimator estimates that the mental health state of the target person improves when detecting that the positive expression score in a latest predetermined period has a rising tendency compared with an ordinary value as the feature associated with the temporal change of the expression. Therefore, the rising of the expression indicating the positive emotion can quantitatively be evaluated, and the improvement of the mental health state can be estimated with high reliability. Alternatively, the health state estimator may select or generate a negative expression score indicating the degree of the negative emotion from the scores of the plurality of kinds of the expressions, and the health state estimator may estimate that the mental health state of the target person improves when detecting that the negative expression score in the latest predetermined period has a lowering tendency compared with the ordinary value as the feature associated with the temporal change of the expression. Therefore, the lowering of the expression indicating the negative emotion can quantitatively be evaluated, and the improvement of the mental health state can be estimated with high reliability.

It may be preferable that the expression recognizer calculates a score in which a degree of each of a plurality of kinds of the expressions is digitized from the image of the target person, and outputs the score of each expression as the expression recognition result, the health state estimator selects or generates a positive expression score indicating the degree of the positive emotion from the scores of the plurality of kinds of the expressions, and the health state estimator estimates that the mental health state of the target person becomes worse when detecting that a fluctuation range of the positive expression score in a latest predetermined period has a decreasing tendency compared with an ordinary value as the feature associated with the temporal change of the expression. Therefore, the decrease of the expression indicating the positive emotion can quantitatively be evaluated, and the worsening of the mental health state can be estimated with high reliability. Alternatively, the health state estimator may select or generate a negative expression score indicating the degree of the negative emotion from the scores of the plurality of kinds of the expressions, and the health state estimator may estimate that the mental health state of the target person becomes worse when detecting that a fluctuation range of the negative expression score in the latest predetermined period has an increasing tendency compared with the ordinary value as the feature associated with the temporal change of the expression. Therefore, the increase of the expression indicating the negative emotion can quantitatively be evaluated, and the worsening of the mental health state can be estimated with high reliability.

It may be preferable that the expression recognizer calculates a score in which a degree of each of a plurality of kinds of the expressions is digitized from the image of the target person, and outputs the score of each expression as the expression recognition result, the health state estimator selects or generates a positive expression score indicating the degree of the positive emotion from the scores of the plurality of kinds of the expressions, and the health state estimator estimates that the mental health state of the target person improves when detecting that a fluctuation range of the positive expression score in a latest predetermined period has an increasing tendency compared with an ordinary value as the feature associated with the temporal change of the expression. Therefore, the increase of the expression indicating the positive emotion can quantitatively be evaluated, and the improvement of the mental health state can be estimated with high reliability. Alternatively, the health state estimator may select or generate a negative expression score indicating the degree of the negative emotion from the scores of the plurality of kinds of the expressions, and the health state estimator may estimate that the mental health state of the target person improves when detecting that a fluctuation range of the negative expression score in the latest predetermined period has a decreasing tendency compared with the ordinary value as the feature associated with the temporal change of the expression. Therefore, the decrease of the expression indicating the negative emotion can quantitatively be evaluated, and the improvement of the mental health state can be estimated with high reliability.

It may be preferable that the expression recognizer calculates a score in which a degree of each of a plurality of kinds of the expressions is digitized from the image of the target person, and outputs the score of each expression as the expression recognition result, the health state estimator selects or generates a positive expression score indicating the degree of the positive emotion from the scores of the plurality of kinds of the expressions, and the health state estimator estimates that the mental health state of the target person becomes worse when detecting that an evening score tends to be relatively higher than a morning score in a daily fluctuation of the positive expression score as the feature associated with the temporal change of the expression. Alternatively, the health state estimator may select or generate a negative expression score indicating the degree of the negative emotion from the scores of the plurality of kinds of the expressions, and the health state estimator estimates that the mental health state of the target person becomes worse when detecting that the evening score tends to be relatively lower than the morning score in a daily fluctuation of the negative expression score as the feature associated with the temporal change of the expression. Therefore, the appearance of the symptom that the feeling is down in the morning while the feeling is good in the evening can quantitatively be evaluated, and the worsening of the mental health state can be estimated with high reliability.

It may be preferable that the health state estimator estimates that the mental health state of the target person becomes worse when detecting a change of an appearance ratio of a plurality of kinds of the expressions as the feature associated with the temporal change of the expression. This is because such a change of an emotional expression (a change of personality) that a person becomes easily angry is generated in mental diseases such as dementia.

It may be preferable that the expression recognizer calculates a score in which a degree of each of the plurality of kinds of the expressions is digitized from the image of the target person, and outputs the score of each expression as the expression recognition result, and the health state estimator estimates that the mental health state of the target person becomes worse when detecting that a difference between a score mean in a latest predetermined period and an ordinary value is larger than a threshold with respect to a part of or all the plurality of kinds of the expressions as the feature associated with the temporal change of the expression. Therefore, the change of the appearance ratio of the expression can quantitatively be evaluated, and the worsening of the mental health state can be estimated with high reliability.

It may be preferable that the expression recognizer calculates a score in which a degree of each of the plurality of kinds of the expressions is digitized from the image of the target person, and outputs the score of each expression as the expression recognition result, and the health state estimator estimates that the mental health state of the target person becomes worse when detecting that a fluctuation range of a score of a certain expression in a latest predetermined period has an increasing tendency compared with an ordinary value as the feature associated with the temporal change of the expression. Therefore, the appearance of the symptom that undulation of the feeling is intensified can quantitatively be evaluated, and the worsening of the mental health state can be estimated with high reliability.

It may be preferable that the ordinary value is a value that is statistically obtained from the time-series data of the target person, the time-series data being stored in the storage. Using a value statistically obtained from the time-series data of the target person as the ordinary value, the temporal change of the expression can be evaluated based on the personality (such as an original face, an expression in an ordinary state, and an emotional expression) of the expression of the target person. Therefore, degradation of estimation accuracy due to an individual difference can be prevented to enhance the reliability of estimation processing.

It may be preferable that the health state estimator estimates the mental health state using the time-series data of the target person for a plurality of days. It may be preferable that the latest predetermined period is a period longer than one day. Because even a healthy person feels down or good or changes the feeling, it is difficult to estimate the mental health state of the person only from the change of the expression in a period of several hours to one day (the reliability is low even if the mental health state of the person can be estimated). It may be preferable that the expression change is evaluated every day, every week, every month, or every year depending on “the feature associated with the temporal change of the expression” to be detected. Accordingly, for example, the latest predetermined period may be set to such a period as several days, one week to several weeks, one month to several months, and one year to several years.

One or more embodiments of the present invention can also be understood as a health care assisting device including at least a part of the above configurations or functions. One or more embodiments of the present invention can also be understood as a health care assisting method including at least a part of the above pieces of processing, a program causing a computer to perform the health care assisting method, or a computer-readable recording medium in which the program is non-transiently stored. One or more embodiments of the present invention can be implemented by a combination of the configurations or the pieces of processing as long as technical inconsistency is not generated.

One or more embodiments of the present invention can estimate the mental health state of the person based on the facial expression recognized from the image, and provide the information useful for the mental health care.

### BRIEF DESCRIPTION OF THE DRAWINGS

FIG. 1 is a view illustrating a configuration example of a health care assisting device;

FIG. 2 is a flowchart illustrating a flow of expression recognition processing;

FIG. 3 is a view illustrating an example of time-series data of an expression recognition result stored in a storage;

FIG. 4 is a view illustrating an example of a positive expression score of a mental disease;

FIGS. 5A and 5B are views illustrating an estimation logic of a health state estimator in a specific example (1);

FIGS. 6A and 6B are views illustrating the estimation logic of the health state estimator in a specific example (2);

FIGS. 7A and 7B are views illustrating the estimation logic of the health state estimator in a specific example (3);

FIGS. 8A and 8B are views illustrating the estimation logic of the health state estimator in a specific example (4); and

FIGS. 9A and 9B are views illustrating the estimation logic of the health state estimator in a specific example (5).

### DETAILED DESCRIPTION

Hereinafter, embodiments of the present invention will be described in detail with reference to the drawings. However, unless otherwise noted, the present invention is not limited to sizes, materials, shapes, and relative dispositions of components described in the following embodiment.

(Device Configuration)

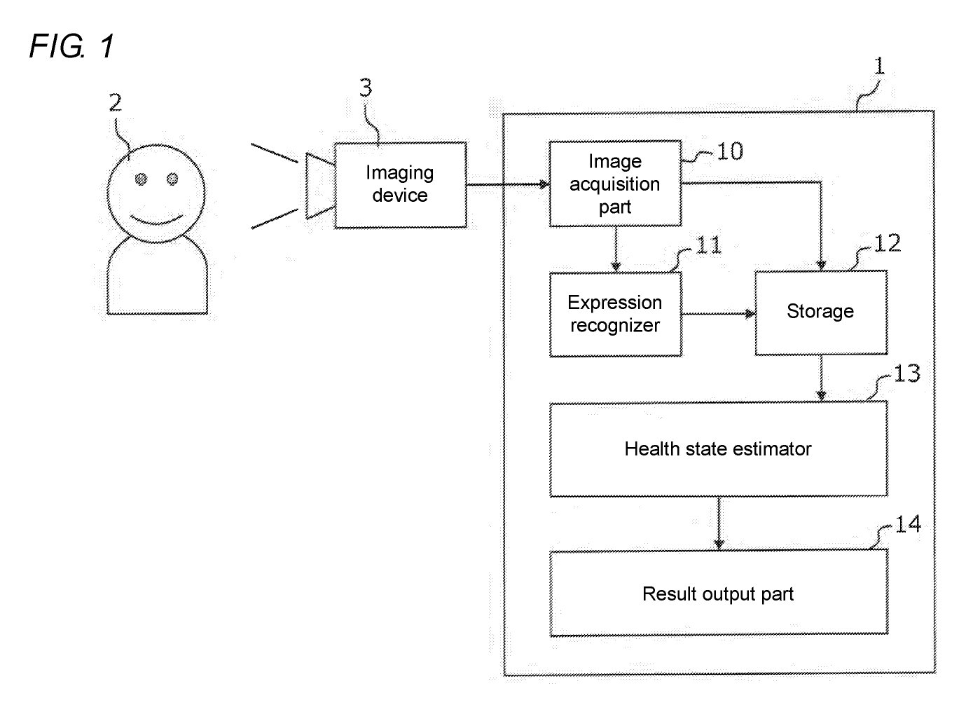

FIG. 1 is a view illustrating a configuration example of a health care assisting device according to an embodiment of the present invention. A health care assisting device 1 analyzes an image in which an object person 2 is photographed, and provides information useful for mental health care of the object person 2. The health care assisting device 1 can be applied to various purposes such as self-check of the target person, a diagnostic tool for a specialist such as a medical doctor and counselor, and mental health case in a corporation or a school.

The health care assisting device 1 in FIG. 1 includes an image acquisition part 10, an expression recognizer 11, a storage 12, a health state estimator 13, and a result output part 14 as a main configuration.

The image acquisition part 10 has a function of acquiring the image from an imaging device 3. In the embodiment, a plurality of images in which a face of the object person 2 is photographed in time series are sequentially captured from the imaging device 3. The imaging device 3 includes a monochrome or color camera. In FIG. 1, the imaging device 3 is provided separately from the health care assisting device 1. Alternatively, the imaging device 3 may be mounted on the health care assisting device 1. The expression recognizer 11 has a function of recognizing a facial expression from the image through image sensing processing. The storage 12 has a function of storing an expression recognition result output from the expression recognizer 11 as time-series data. The health state estimator 13 has a function of detecting a feature associated with a time change of an expression of the object person 2 from the time-series data stored in the storage 12, and estimating a mental health state of the object person 2 based on the detected feature. The result output part 14 has a function of outputting an estimation result of the health state estimator 13.

An interval at which the image of the target person 2 is photographed or captured may properly be set in response to use or usage environment of the health care assisting device 1. For example, in order to evaluate a daily fluctuation of the facial expression or a change of the facial expression for a plurality of days (for example, several days, several weeks, or several months), the image may be photographed or captured at a frequency of once per several seconds to several minutes or a frequency of once per several tens of minutes to several hours. In the case that the target person 2 or the imaging device 3 always exists in a visual field, the photographing can be performed at constant time intervals or a fixed time. In the case that the target person 2 or the imaging device 3 does not always exist in the visual field, for example, the photographing is performed when a human sensor can detect the target person 2, or the target person 2 performs self-photographing at a predetermined frequency.

The health care assisting device 1 may include a computer including a CPU (processor), a memory, an auxiliary storage device, an input device, a display device, and a communication device. A program stored in the auxiliary storage device is loaded on the memory, and the CPU executes the program, thereby implementing each function of the health care assisting device 1. However, a part of or all the functions of the health care assisting device 1 can also be implemented by a circuit such as an ASIC and an FPGA. Alternatively, a part of the functions (for example, the functions of the expression recognizer 11, storage 12, and emotion estimator 13) of the health care assisting device 1 may be implemented by cloud computing or distributed computing.

(Time-Series Data of Expression Recognition Result)

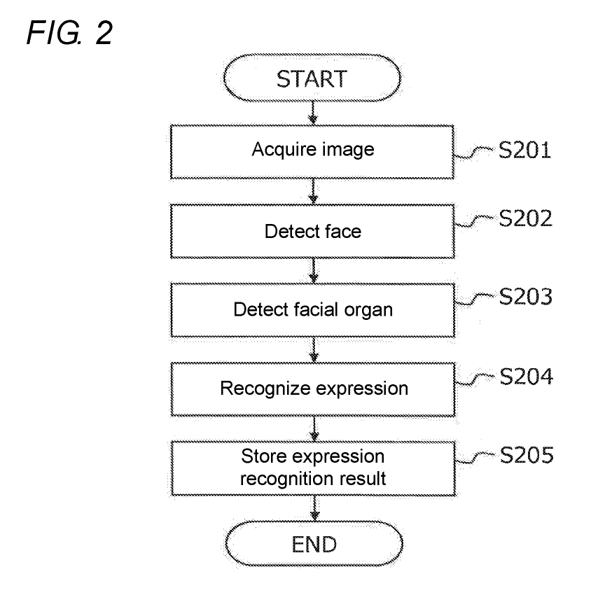

A flow of expression recognition processing performed by the health care assisting device 1 will be described with reference to FIG. 2. FIG. 2 is a flowchart illustrating the flow of the expression recognition processing. The expression recognition processing in FIG. 2 is performed every time the target person 2 is photographed by the imaging device 3.

The image acquisition part 10 acquires the image, in which the object person 2 is photographed, from the imaging device 3 in Step S201. Desirably the image in which a front face of the object person 2 is photographed as much as possible is acquired in order to recognize the facial expression. Then, the expression recognizer 11 detects the face from the image (Step S202), and detects a facial organ (such as eyes, eyebrows, a nose, and a mouth) (Step S203). Because any algorithm including a well-known technique may be used in the face detection and the facial organ detection, the detailed description is omitted.

The expression recognizer 11 recognizes the facial expression of the object person 2 using detection results in Steps S202 and S203 (Step S204). In the embodiment, based on an expression analysis of Paul Ekman, the facial expression is classified into seven kinds including “anger”, “disgust”, “fear”, “happiness”, “sadness”, “surprise”, and “straight face (expressionless)”. A score is output as the expression recognition result such that degrees (also referred to as expression-likeness or an expression degree) of the seven kinds of the expressions become 100 in total. The score of each expression is also referred to as an expression component value. In the following description, sometimes numbers are added to the seven expressions, and the scores of the expressions are also written as S₁ and S₇.

1: anger

2: disgust

3: fear

4: happiness

5: sadness

6: surprise

7: straight face

Any algorithm including a well-known technique may be used in the expression recognition in Step S204. An example of the expression recognition processing will be described below. The expression recognizer 11 extracts a feature amount associated with the relative position or shape of the facial organ based on positional information on the facial organ. For example, a Haar-like feature amount, a distance between feature points, and the Fourier descriptor can be used as the feature amount. The expression recognizer 11 inputs the extracted feature amount to a classifier for each of the seven kinds of the facial expressions, and calculates the degrees of the expressions. Each classifier can be generated by learning in which a sample image is used. Finally, the expression recognizer 11 performs normalization such that output values of the seven classifiers become 100 in total, and outputs the scores (expression component values) of the seven kinds of the expressions.

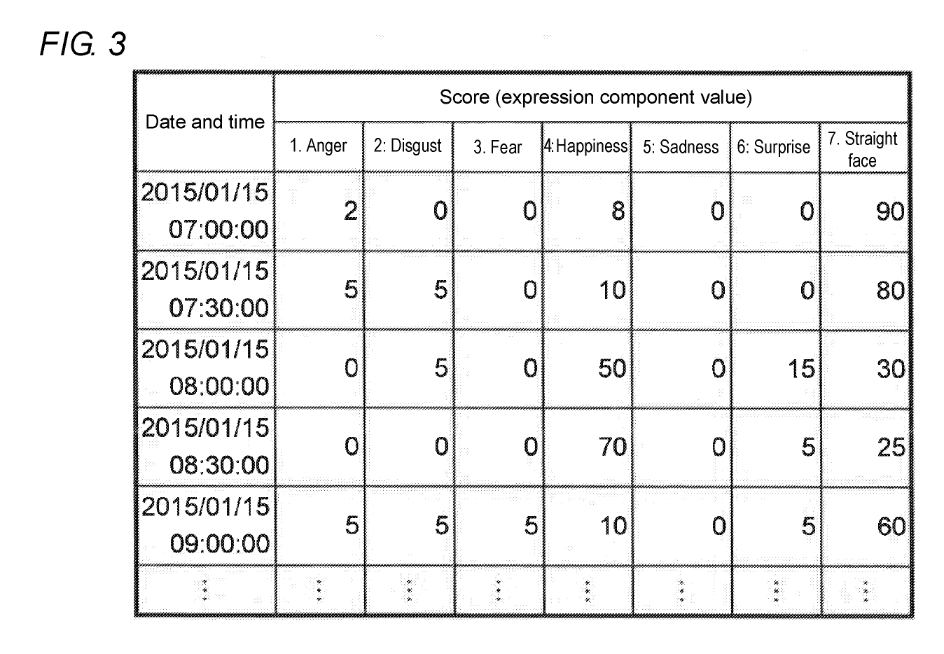

The expression recognizer 11 stores the expression recognition result in a database of the storage 12 together with time stamp information (Step S205). FIG. 3 illustrates an example of the time-series data of the expression recognition result stored in the storage 12, and each line indicates the expression recognition result obtained from one facial image.

(Estimation of Mental Health State)

Processing of estimating the mental health state will be described below. In the health care assisting device 1 of the embodiment, the health state estimator 13 detects the feature associated with the temporal change of the facial expression of the target person 2 from the time-series data of the expression recognition result (the processing is also referred to as a “time-series analysis of expression data”), and estimates the mental health state of the target person 2 based on the detected feature. There are various “features associated with the temporal change of the facial expression” that can be detected by the time-series analysis of the expression data. The specific examples (1) to (5) will be described below.

(1) A Decrease of the Expression Indicating the Positive Emotion (the Lowering of the Score)

Typical symptoms of the “depression” that is one of the positive emotions include the following items.
to feel down (depressive feeling)
to be uninterested, and too hard to enjoy
to feel tired, or to be in low spirit
to be not able to focus on work or housekeeping or to make decision
to be slow in acting or speaking, or to be irritated or restless
to have a poor (good) appetite, or to gain or lose weight
to be not able to get sleep, or to awake in the night or early morning
to feel being of no worth or an apology
to sometimes feel to wand to disappear from the world
   Many of these symptoms generate the change of the facial expression, and appear as the sign such as “dark expression”, “decreased smile”, and “poor expression”.

Therefore, the specific example (1) focuses on “the decrease of the expression indicating the positive emotion” as the feature associated with the temporal change of the expression. Particularly, in the embodiment, a “positive expression score Sₚₒₛ” that is of an index indicating a degree of the positive emotion is defined as follows.

Sₚₒₛ=happiness score S₄−anger score S₁−sadness score S₅

The positive expression score Sₚₒₛ is designed so as to indicate a higher value in the case that target person 2 feels good and positive, and to indicate a lower value in the case that target person 2 feels down and irritable.

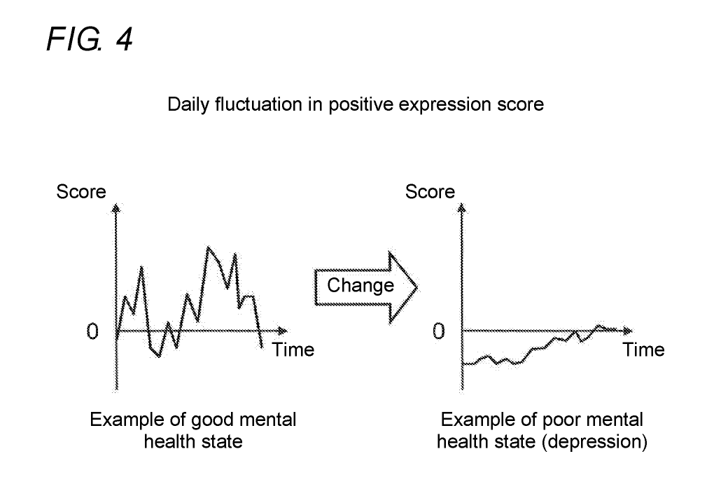

FIG. 4 illustrates an example of the daily fluctuation of the positive expression score Sₚₒₛ. For a person who is in the good mental health state, because the emotion changes usually in a day, the positive emotion score Sₚₒₛ fluctuates largely as illustrated in the left graph of FIG. 4. On the other hand, for a person having depression, the positive expression score Sₚₒₛ tends to decrease as a whole or a fluctuation range of the positive expression score Sₚₒₛ tends to be reduced as illustrated in the right graph of FIG. 4. There is a characteristic depression symptom that the person feels down in the morning while feeling gradually better in the evening. Accordingly, the score change is detected by the time-series analysis of the expression data, which allows the change (worsening) of the mental health state to be discovered as the indication or symptom of the mental disease (particularly, the depression). As used herein, “the indication of the mental disease” means a symptom that appears before the mental disease or at an extremely early stage of the mental disease.

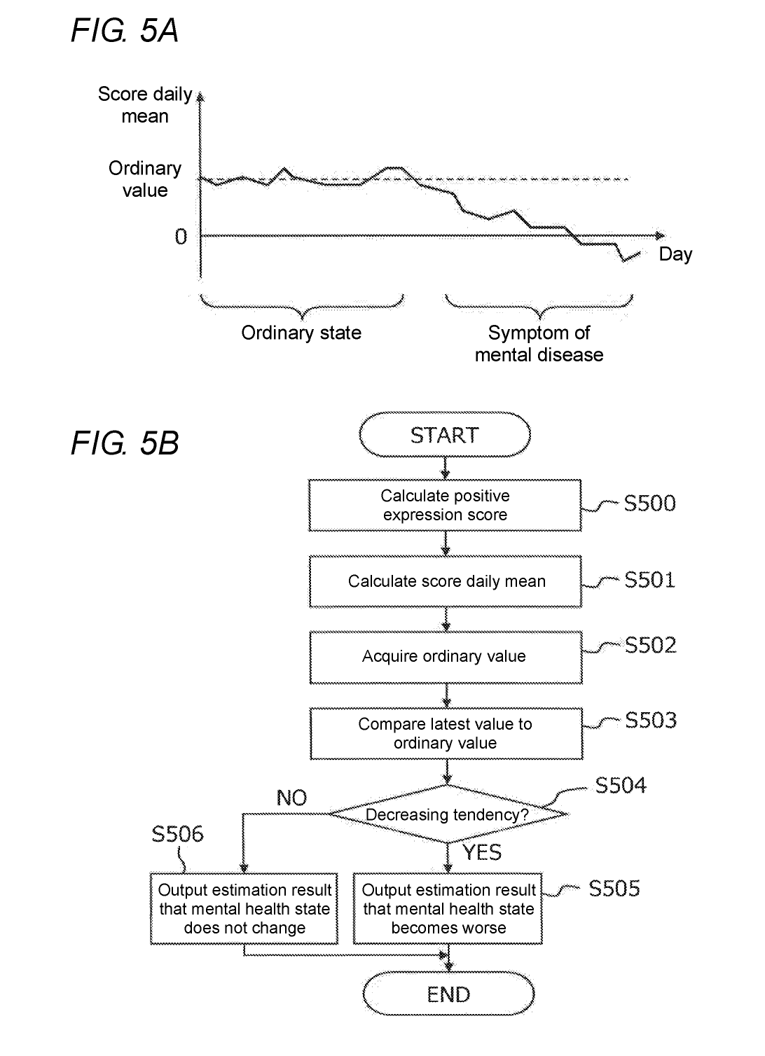

The processing of the health state estimator 13 will be described with reference to FIGS. 5A and 5B. FIG. 5A is a graph illustrating a change of the positive expression score Sₚₒₛ of the target person 2. In FIG. 5A, a horizontal axis indicates a day, and a vertical axis indicates a daily mean (hereinafter, referred to as a score daily mean DSₚₒₛ) of the positive expression score Sₚₒₛ. The latest score daily mean DSₚₒₛ indicates a lowering tendency, and the indication of the mental disease (depression) appears. FIG. 5B illustrates a processing flow of the health state estimator 13 in the specific example (1).

In Step S500, the health state estimator 13 reads the time-series data in a necessary period (for example, past one month) from the storage 12, and calculates the positive expression score Sₚₒₛ of each piece of time-series data. The health state estimator 13 calculates the daily mean DSₚₒₛ of the positive expression score Sₚₒₛ (Step S501). The health state estimator 13 acquires an ordinary value RDS of the score daily mean DSₚₒₛ (Step S502). The ordinary value RDS is a score daily mean DSₚₒₛ when the mental health state is in an ordinary state. At this point, it is assumed that a mean for past one month of the score daily mean DSₚₒₛ of the target person 2 is used as the ordinary value RDS.

Then, the health state estimator 13 compares the score daily mean DSₚₒₛ to the ordinary value RDS in the latest predetermined period (for example, several days to one week), and determines whether the latest value indicates the lowering tendency with respect to the ordinary value RDS (Steps S503 and S504). At this point, assuming that σ is a standard deviation for past one month of the score daily mean DSₚₒₛ, a determination that the latest value has the lowering tendency is made in the case that the score daily mean DSₚₒₛ satisfying the following expression continues for a predetermined period (for example, several days to one week).

DSₚₒₛ<RDS−n×σ

Where n is a parameter adjusting detection sensitivity. For example, n is set to a value of 1 to 3.

When the score lowering tendency is detected in Step S504, the health state estimator 13 outputs an estimation result that the mental health state of the target person 2 becomes worse (Step S505). When the score lowering tendency is not detected, the health state estimator 13 outputs the estimation result that the mental health state of the target person 2 does not change (Step S506). Therefore, the indication or symptom (for example, a possibility of the depression) of the mental disease of the target person 2 can automatically and early be discovered.

(2) A Decrease of the Expression Indicating the Positive Emotion (a Decrease in Fluctuation Range of the Score)

As illustrated in FIG. 4, the fluctuation range of the positive expression score Sₚₒₛ tends to decrease in the depressive state. Therefore, in the specific example (2), the indication or symptom of the mental disease (particularly, the depression) is discovered by detecting the lowering of a variance of the positive expression score Sₚₒₛ.

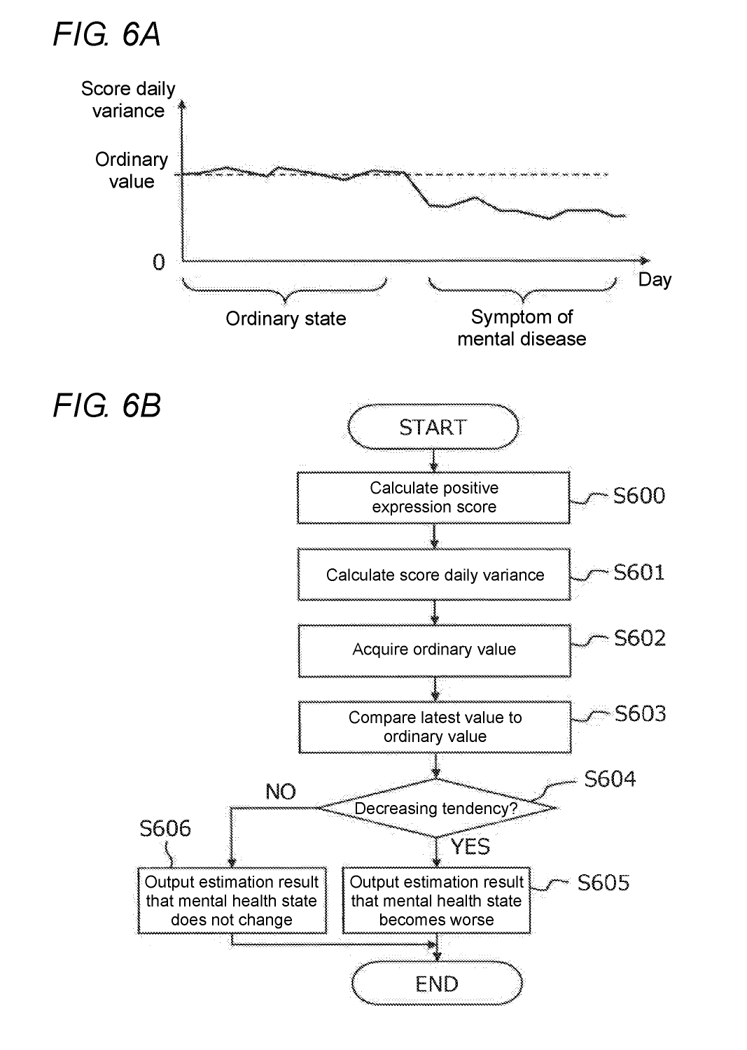

The processing of the health state estimator 13 will be described with reference to FIGS. 6A and 6B. FIG. 6A is a graph illustrating the change of the positive expression score Sₚₒₛ of a certain target person 2. In FIG. 6A, a horizontal axis indicates a day, and a vertical axis indicates a daily variance (hereinafter, referred to as a hereinafter, referred to as a score daily variance VSₚₒₛ) of the positive expression score Sₚₒₛ. The latest score daily variance VSₚₒₛ indicates a lowering tendency, and the indication of the mental disease (depression) appears. FIG. 6B illustrates the processing flow of the health state estimator 13 in the specific example (2).

In Step S600, the health state estimator 13 reads the time-series data in a necessary period (for example, past one month) from the storage 12, and calculates the positive expression score Sₚₒₛ of each piece of time-series data. The health state estimator 13 calculates the score daily variance VSₚₒₛ of the positive expression score Sₚₒₛ (Step S601). The health state estimator 13 acquires an ordinary value RVS of the score daily variance VSₚₒₛ (Step S602). The ordinary value RVS is a score daily variance VSₚₒₛ when the mental health state is in the ordinary state. At this point, it is assumed that a mean for past one month of the score daily variance VSₚₒₛ of the target person 2 is used as the ordinary value RVS.

Then, the health state estimator 13 compares the score daily variance VSₚₒₛ to the ordinary value RVS in the latest predetermined period (for example, several days to one week), and determines whether the latest value indicates the lowering tendency with respect to the ordinary value RVS (Steps S603 and S604). At this point, assuming that σ is a standard deviation for past one month of the score daily variance VSₚₒₛ, a determination that the latest value has the lowering tendency (that is, the score fluctuation range has a decreasing tendency) is made in the case that the score daily variance VSₚₒₛ satisfying the following expression continues for a predetermined period (for example, several days to one week).

VSₚₒₛ<RVS−n×σ

Where n is a parameter adjusting the detection sensitivity. For example, n is set to a value of 1 to 3.

When the decreasing tendency of the score fluctuation range is detected in Step S604, the health state estimator 13 outputs the estimation result that the mental health state of the target person 2 becomes worse (Step S605). On the other hand, when the decreasing tendency of the score fluctuation range is not detected, the health state estimator 13 outputs the estimation result that the mental health state of the target person 2 does not change (Step S606). Therefore, the indication or symptom (for example, the possibility of the depression) of the mental disease of the target person 2 can automatically and early be discovered.

The lowering of the score daily variance is detected in the specific example (2). Alternatively, the decreasing tendency of the score fluctuation range can be grasped by detecting such a decrease in frequency or time that the value of the positive expression score Sₚₒₛ exceeds a threshold.

(3) An Expression Change Between the Morning and the Evening

As illustrated in FIG. 4, there is the characteristic depression symptom that the person feels down in the morning while feeling gradually better in the evening. Therefore, in the specific example (3), the indication or symptom of the mental disease (particularly, the depression) is discovered by evaluating the daily fluctuation of the positive expression score Sₚₒₛ.

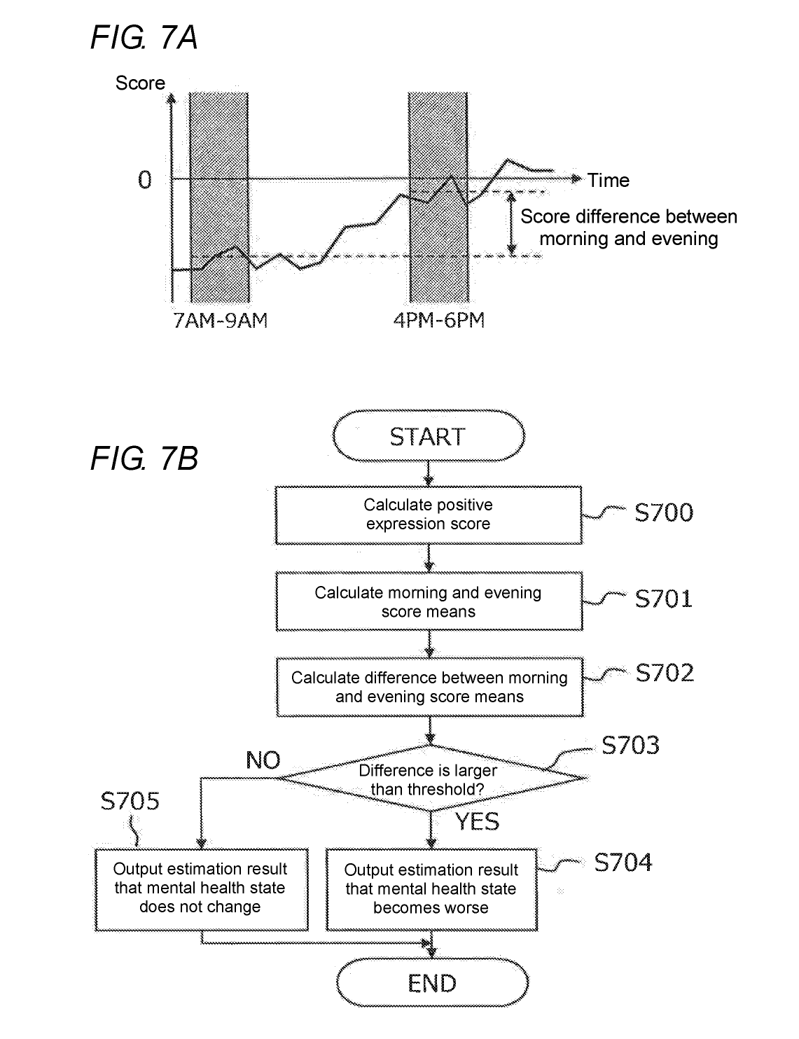

The processing of the health state estimator 13 will be described with reference to FIGS. 7A and 7B. FIG. 7A is a graph illustrating the daily fluctuation in positive expression score Sₚₒₛ that is observed in a person having the depression. In FIG. 7A, a horizontal axis indicates time, and a vertical axis indicates the positive expression score Sₚₒₛ. The positive expression score Sₚₒₛ is the minimum in the morning, increases gradually over time, and becomes relatively higher in the evening compared with the morning score. FIG. 7B illustrates the processing flow of the health state estimator 13 in the specific example (3).

In Step S700, the health state estimator 13 reads the time-series data for one day from the storage 12, and calculates the positive expression score Sₚₒₛ of each piece of time-series data. The health state estimator 13 calculates means of the positive expression scores Sₚₒₛ in the morning and evening (Step S701). At this point, a morning score mean ASₚₒₛ is obtained from the pieces of time-series data of 7 a.m. to 9 a.m., and an evening score mean PSₚₒₛ is obtained from the pieces of time-series data of 4 p.m. to 6 p.m.

Then, the health state estimator 13 calculates a difference ΔSₚₒₛ (=PSₚₒₛ−ASₚₒₛ) between the morning score mean ASₚₒₛ and the evening score mean PSₚₒₛ (Step S702), and compares the difference ΔSₚₒₛ to a threshold Th1 (Step S703). When the difference ΔSₚₒₛ is larger than the threshold Th1 (YES in Step S703), the health state estimator 13 outputs the estimation result that the mental health state of the target person 2 becomes worse (Step S704). On the other hand, when the difference ΔSₚₒₛ is less than or equal to the threshold Th1 (NO in Step S703), the health state estimator 13 outputs the estimation result that the mental health state of the target person 2 does not change (Step S705). Therefore, the indication or symptom (for example, the possibility of the depression) of the mental disease of the target person 2 can automatically and early be discovered.

Only the daily fluctuation for one day is evaluated in the specific example (3). Alternatively, the daily fluctuation for latest several days is evaluate and the determination that the mental health state becomes worse may be made in the case that the daily variance in FIG. 7A continues for several days.

(4) A Change of an Appearance Ratio of the Expression

For the “dementia” that is of one of the mental diseases, examples of action and mental symptom include the following items.
uneasiness and impatient
depressive state
hallucination and delusion
excitement and violence

These symptoms generate a change of an emotional expression (a change of personality), and appear sometimes as the signs such as “becoming easily angry”, “intensifying undulation of the feeling”, and the increase of the disgust or sadness expression”.

Therefore, in the specific example (4), the indication or symptom of the mental disease (particularly, the dementia) is discovered by evaluating the change of the appearance ratio of seven expressions.

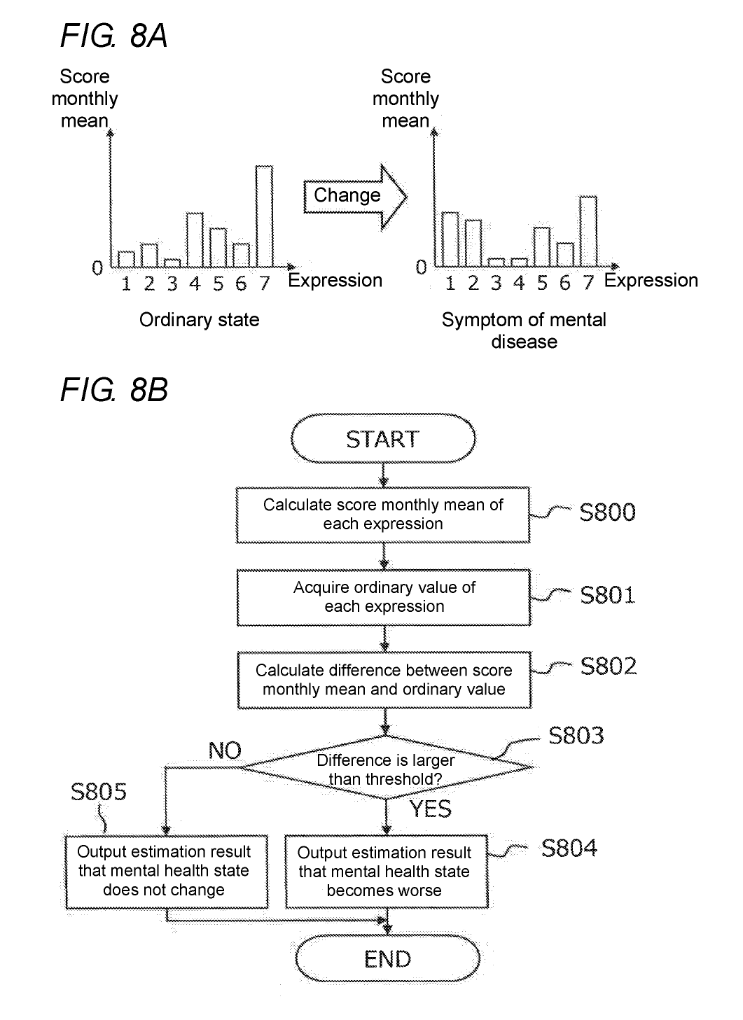

The processing of the health state estimator 13 will be described with reference to FIGS. 8A and 8B. FIG. 8A is a graph illustrating the appearance ratio of the seven expressions. In FIG. 8A, a horizontal axis indicates a number of the expression (1: anger, 2: disgust, 3: fear, 4: happiness, 5: sadness, 6: surprise, 7: straight face), and a vertical axis indicates monthly means of scores S₁ to S₇. As illustrated in the left graph of FIG. 8A, the many expressions of “4: happiness” or “5: sadness” are observed for the good mental health state. As illustrated in the right graph of FIG. 8A, due to the dementia symptom, the expression of “4: happiness” decreases while the expressions of “1: anger” and “2: disgust” increase. FIG. 8B illustrates the processing flow of the health state estimator 13 in the specific example (4).

The health state estimator 13 reads the time-series data in the necessary period (for example, past two months) from the storage 12, and calculates monthly means (score monthly means MS₁ to MS₇) of the scores S₁ to S₇ of the seven expressions (Step S800). The health state estimator 13 acquires ordinary values RMS₁ to RMS₇ of the score monthly means MS₁ to MS₇ (Step S801). The ordinary values RMS₁ to RMS₇ are values of the score monthly means MS₁ to MS₇ when the mental health state is in the ordinary state. At this point, it is assumed that the past (for example, before one month or more) score monthly means MS₁ to MS₇ of the target person 2 are used as the ordinary values RMS₁ to RMS₇.

Then the health state estimator 13 calculates a difference ΔS between the score monthly means MS₁ to MS₇ and the ordinary values RMS₁ to RMS₇ using the following equation (Step S803).

ΔS=Σ(∥RMSᵢ−Msᵢ|) (i=1, 2, . . . , 7)

The difference ΔS is an index indicating magnitude of the change of the appearance ratio of the expressions between the latest predetermined period (in this case, one month) and ordinary state.

The health state estimator 13 compares the difference ΔS to a threshold Th2 (Step S803). When the difference ΔS is larger than a threshold Th2 (YES in Step S803), the health state estimator 13 outputs the estimation result that the mental health state of the target person 2 becomes worse (Step S804). On the other hand, when the difference ΔS is less than or equal to the threshold Th2 (NO in Step S803), the health state estimator 13 outputs the estimation result that the mental health state of the target person 2 does not change (Step S805). Therefore, the indication or symptom (for example, the possibility of the depression) of the mental disease of the target person 2 can automatically and early be discovered.

The monthly mean is evaluated in the specific example (4). Alternatively, the evaluation may be performed using the mean of a plurality of days or the means of a plurality of weeks. Alternatively, not all the scores of the seven expressions, but only the scores of the expressions (for example, “anger”, “disgust”, and “happiness”) in which an appearance frequency increases due to the dementia may be used in the evaluation.

(5) The Intensification of the Undulation of the Emotion

As described above, there is the intensification of the undulation of the emotion as the dementia symptom. Therefore, in the specific example (5), the indication or symptom of the dementia is discovered by detecting the increase of the fluctuation range of the score of one of the seven expressions.

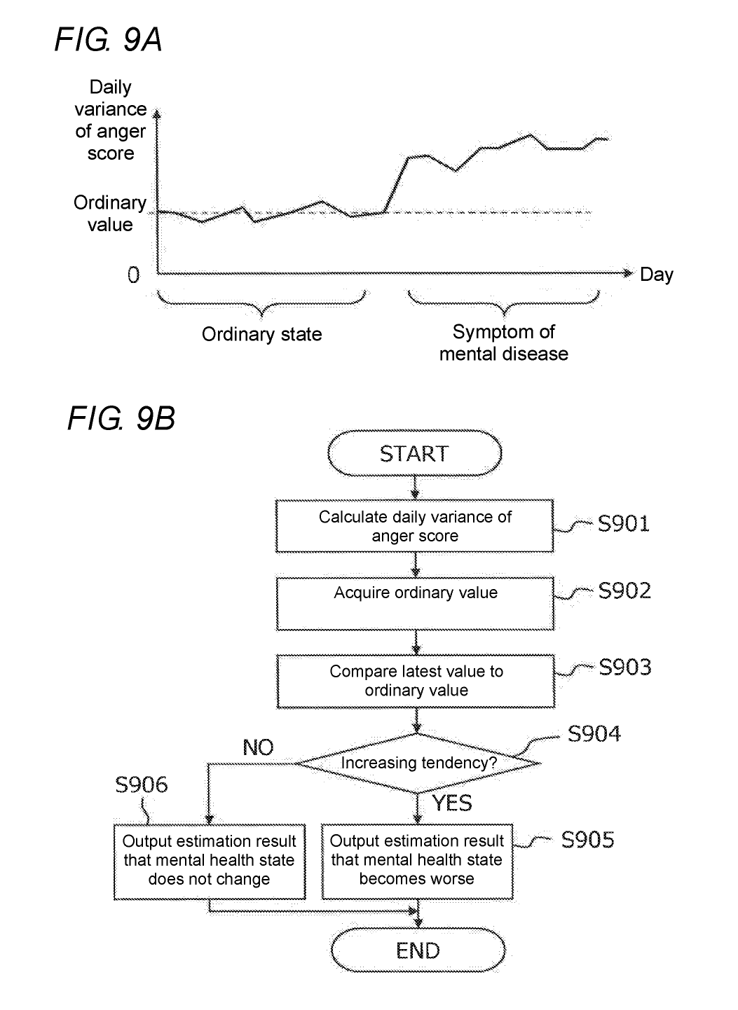

The processing of the health state estimator 13 will be described with reference to FIGS. 9A and 9B. FIG. 9A is a graph illustrating the change of the anger score S₁ of a certain target person 2. In FIG. 9A, a horizontal axis indicates a day, and a vertical axis indicates the daily variance (hereinafter, referred to as a score daily variance VS₁) of the anger score S₁. As can be seen from FIG. 9A, the latest score daily variance VS₁ has the rising tendency, and the indication of the mental disease (dementia) appears. FIG. 9B illustrates the processing flow of the health state estimator 13 in the specific example (5).

In Step S901, the health state estimator 13 reads the time-series data in the necessary period (for example, past one month) from the storage 12, and calculates the daily variance VS₁ of anger score S₁. The health state estimator 13 acquires an ordinary value RVS₁ of the score daily variance VS₁ (Step S902). The ordinary value RVS₁ is a value of the score daily variance VS₁ when the mental health state is in the ordinary state. At this point, it is assumed that the mean of the score daily variances VS₁ for past one month of the target person 2 is used as the ordinary value RVS₁.

Then, the health state estimator 13 compares the score daily variance VS₁ to the ordinary value RVS₁ in the latest predetermined period (for example, several days to one week), and determines whether the latest value has the rising tendency with respect to the ordinary value RVS₁ (Steps S903 and S904). At this point, assuming that σ is a standard deviation for past one month of the score daily variance VS₁, a determination that the latest score daily variance has the rising tendency (that is, the fluctuation range of the anger score has an increasing tendency) is made in the case that the score daily variance VS₁ satisfying the following expression continues for a predetermined period (for example, several days to one week).

VS₁>RVS₁+n×σ

Where n is a parameter adjusting the detection sensitivity. For example, n is set to a value of 1 to 3.

When the increasing tendency of the fluctuation range of the anger score is detected in Step S904, the health state estimator 13 outputs the estimation result that the mental health state of the target person 2 becomes worse (Step S905). On the other hand, when the increasing tendency of the fluctuation range of the anger score is not detected, the health state estimator 13 outputs the estimation result that the mental health state of the target person 2 does not change (Step S906). Therefore, the indication or symptom (for example, the possibility of the depression) of the mental disease of the target person 2 can automatically and early be discovered.

The rising of the score daily variance is detected in the specific example (5). Alternatively, the increasing tendency of the fluctuation range of the anger score can be grasped by detecting such an increase in frequency or time that the value of the anger score S₁ exceeds a threshold. Not only the anger score S₁ but also the variance or fluctuation range of the score (for example, disgust score S2) of another expression may be evaluated.

The health state estimator 13 of the embodiment includes at least one estimation logic of the specific examples (1) to (5). However, these estimation logics are only an example of the processing of estimating the mental health state based on the feature associated with the temporal change of the expression, and another estimation logic may be mounted on the health state estimator 13.

(Output of Estimation Result)

When the health state estimator 13 obtains the estimation result, the result output part 14 outputs information on the mental health state of the target person 2 based on the estimation result. Any information can be output to any device by any method, and the estimation result can properly be designed based on the application or configuration of the health care assisting device 1. For example, the result output part 14 can display the image or message on the display device, output audio information to a speaker, or transmit information to an external device (such as smartphone, another computer, and an external storage). Any information such as a possibility of the mental disease, a measure, and advice may be output as long as the information is useful for the mental health care. The graph illustrating the temporal change of the expression may be output as illustrated in FIGS. 5A, 6A, 7A, 8A, and 9A. The good or bad and the tendency of the mental health state can intuitively be understood by viewing the graph (temporal change).

In the case that the health state estimator 13 includes the plurality of estimation logics, the result output part 14 may separately output the estimation results obtained by the estimation logics, or output a result in which the estimation results of the estimation logics are integrated (for example, TRUE (=the mental health state becomes worse) is output when a determination of TRUE is made in one of the estimation logics, or TRUE is output when the determination of TRUE is made in all the estimation logics).

(Application Examples of Health Care Assisting Device)

For example, the following items can be considered as an application example of the health care assisting device 1.

(Application Example 1) Health Care in Office

The face of the target person (such as a desk worker) at work is periodically photographed by a sensor installed on a desk or a ceiling. The health care assisting device 1 (server) collects the image from each sensor through a LAN, estimates the expression recognition and health state of each target person, and accumulates the estimation result. When the indication of the mental disease is detected, a superior is notified of the information on the indication of the mental disease. The superior can confirm the mental health state of a subordinate by accessing the health care assisting device 1. In this system, because the superior can confirm the mental health state of the subordinate in real time as objective information, the lowering of the mental health or the indication of the mental disease can quickly be perceived. The superior can quickly take proper action by talking with the subordinate, reducing a burden on work, cooperating early with a health care room or the like.

(Application Example 2) Watch for Aged Person

The target person is an aged person who lives alone. The face of the target person is periodically photographed by a sensor installed in a television set or a kitchen. The health care assisting device 1 collects the image from the sensor installed in house, estimates the expression recognition and health state of the target person, and uploads the estimation result to a cloud server. A family who lives separately from the target person, a social worker, or a medical doctor can check the mental health state of the target person by accessing the cloud server when needed. Alternatively, a notification of the mental health state of the target person is received from the cloud server. In this system, the mental health state of the aged person who lives alone can easily be checked from a distant place, and the indication of the dementia can early be discovered.

(Application Example 3) Self-Diagnosis at Home

The face of the target person is periodically photographed by a sensor installed in a dresser or a washstand. The health care assisting device 1 (for example, an application of the smartphone) collects the image from the sensor, estimates the expression recognition and health state of the target person, and accumulates the estimation result. In this system, a user can check the own mental health state on the smartphone when needed.

The configuration of the embodiment has the following advantages. Because the health care assisting device 1 focuses on the feature associated with the temporal change of the facial expression, the change (improve or worse) of the mental health state that appears as the change of the facial expression can be detected, and the high-reliability estimation result can be obtained compared with the case that the estimation is performed using only the facial expression in one image. Because the high-reliability estimation result is automatically and early obtained, the useful information can properly be provided according to the mental health state of the target person, and the mental health care of the target person can properly be assisted. As described in the specific examples (1) to (5), the temporal change of the expression is quantitatively evaluated based on the time-series data of the score in which the seven expressions are digitized, so that the worsening of the mental health state that leads to the mental diseases such as the depression and the dementia can be estimated with high reliability. Using the value (in the above example, the mean) statistically obtained from the time-series data of the target person as the ordinary value, the temporal change of the expression can be evaluated based on the personality (such as the original face, the expression in the ordinary state, and the emotional expression) of the expression of the target person. Therefore, the degradation of the estimation accuracy due to the individual difference can be prevented to enhance the reliability of the estimation processing.

The configuration of the embodiment illustrates only one specific example of the present invention, but does not limit the scope of the present invention. Various specific configurations can be made without departing from the technical thought of the present invention. For example, in the embodiment, the expressions are classified into seven kinds. Alternatively, another expression classification may be used. In the embodiment, the positive expression score is generated from the happiness score, the anger score, and the sadness score. However, the definition of the positive expression score is not limited to that of the embodiment. For example, one (for example, happiness score) of the seven expressions may directly be selected as the positive expression score. Although the depression and the dementia are cited as an example of the mental disease in the embodiment, the method of the present invention is effective for the mental disease in which the indication (sign) appears in the facial expression. Examples of the possible mental diseases include dissociative disorder, maladjustment, schizophrenia, panic disorder, and anxiety disorder. Any technique such as a regression analysis, a frequency analysis, and trend estimation may be applied to the time-series analysis of the expression data. In the embodiment, the mean is obtained as the ordinary value. Alternatively, preferably another statistical value (such as a median and a mode) is used as long as the statistical value is statistically obtained from the time-series data of the target person.

The expression data period used in the time-series analysis can appropriately be set every day, every week, every month, or every year according to the estimation logic, the feature of the temporal change of the expression, and the kind of the indication or symptom of the mental disease. In the specific example (1), the expressions of the latest several days to one week are set to the evaluation target, and compared to the statistically-obtained ordinary value. The period during which the evaluation target is sampled can be set to several days, one week to several weeks, one month to several months, or one year to several years. Similarly the period during which the ordinary value is sampled can be set to several days, one week to several weeks, one month to several months, or one year to several years. For example, a determination that “the mental health state degrades” is made when the positive expression score for the latest one month is lower than the older score (the score is lowered or the fluctuation range decreases), and the determination that “there is a high possibility of the depression” is made when the lower state of the positive expression score continues for three months.

In some of the mental diseases, the symptom has a seasonal fluctuation. The ordinary value, which is obtained from the past data in the same season as the period in which the evaluation target is sampled, may be sampled in the case that the symptom has the seasonal fluctuation. For example, in the case that progress of the symptom of the dementia is evaluated in units of years, it is conceivable that the expression for the latest one month of this year is compared to the expressions of the same months in the past years.

In the specific example (1), the determination that “the mental health state degrades” is made in the case that the positive expression score Sₚₒₛ is detected to be lower than the ordinary value. On the contrary, the determination that “the mental health state degrades” can be made when the positive expression score Sₚₒₛ is detected to be higher than the ordinary value. For example, assuming that DSₚₒₛ is the score daily mean, that RDS is the ordinary value, that σ is the standard deviation for past one month of the score daily mean DSₚₒₛ, and that n is a parameter adjusting detection sensitivity, the determination that the latest value has the rising tendency can be made in the case that the score daily mean satisfying the following expression continues for a predetermined period.

DSₚₒₛ>RDS+n×σ

Similarly the determination that “the mental health state improves” may be made in the case that the increase of the fluctuation range of the positive expression score Sₚₒₛ is detected compared with that of the ordinary value (past statistical value).

In addition to the positive expression score Sₚₒₛ, attention may be paid to the temporal change of a negative expression score S_{neg} in which the degree of the expression indicating the negative emotion. For example, the expression of the sadness and the expression of the anger corresponds to the expression indicating the negative emotion, and the negative expression score S_{neg} is defined as follows.

S_{neg}=sadness score S₅+anger score S₁

Specifically, the determination that “the mental health state degrades” is made in the case that the negative expression score S_{neg} is higher than the ordinary value or in the case that the fluctuation range of the negative expression score S_{neg} increases compared with that of the ordinary value. On the other hand, the determination that “the mental health state improves” may be made in the case that the negative expression score S_{neg} is lower than the ordinary value or in the case that the fluctuation range of the negative expression score S_{neg} is decreases compared with that of the ordinary value. The daily fluctuation of the negative expression score S_{neg} is evaluated instead of the positive expression score Sₚₒₛ of the specific example (3), and the determination that “the mental health state degrades” is made in the case that the negative expression score S_{neg} is high in the morning while being relatively low in the evening.

1. A health care assisting device configured to assist mental health care of a target person, the health care assisting device comprising:
an image acquisition part configured to acquire a plurality of images in which the target person is photographed in time series;
an expression recognizer configured to recognize a feature of an expression of the target person from the plurality of images acquired by the image acquisition part;
a storage in which expression recognition results of the plurality of images are stores as time-series data;
a health state estimator configured to detect a feature associated with a temporal change of the expression of the target person from the time-series data stored in the storage, and estimate a mental health state of the target person based on the detected feature; and
an output part configured to output information on the mental health state of the target person based on an estimation result of the health state estimator.

2. The health care assisting device according to claim 1, wherein the health state estimator estimates that the mental health state of the target person becomes worse when detecting a decrease of the expression indicating a positive emotion or an increase of the expression indicating a negative emotion as the feature associated with the temporal change of the expression.

3. The health care assisting device according to claim 1, wherein the health state estimator estimates that the mental health state of the target person improves when detecting an increase of the expression indicating a positive emotion or a decrease of the expression indicating a negative emotion as the feature associated with the temporal change of the expression.

4. The health care assisting device according to claim 1, wherein the expression recognizer calculates a score in which a degree of each of a plurality of kinds of the expressions is digitized from the image of the target person, and outputs the score of each expression as the expression recognition result,
the health state estimator selects or generates a positive expression score indicating the degree of the positive emotion or a negative expression score indicating the degree of the negative emotion from the scores of the plurality of kinds of the expressions, and
the health state estimator estimates that the mental health state of the target person becomes worse when detecting that the positive expression score in a latest predetermined period has a lowering tendency compared with an ordinary value or that the negative expression score in the latest predetermined period has a rising tendency compared with the ordinary value as the feature associated with the temporal change of the expression.

5. The health care assisting device according to claim 1, wherein the expression recognizer calculates a score in which a degree of each of a plurality of kinds of the expressions is digitized from the image of the target person, and outputs the score of each expression as the expression recognition result,
the health state estimator selects or generates a positive expression score indicating the degree of the positive emotion or a negative expression score indicating the degree of the negative emotion from the scores of the plurality of kinds of the expressions, and
the health state estimator estimates that the mental health state of the target person improves when detecting that the positive expression score in a latest predetermined period has a rising tendency compared with an ordinary value or that the negative expression score in the latest predetermined period has a lowering tendency compared with the ordinary value as the feature associated with the temporal change of the expression.

6. The health care assisting device according to claim 1, wherein the expression recognizer calculates a score in which a degree of each of a plurality of kinds of the expressions is digitized from the image of the target person, and outputs the score of each expression as the expression recognition result,
the health state estimator selects or generates a positive expression score indicating the degree of the positive emotion or a negative expression score indicating the degree of the negative emotion from the scores of the plurality of kinds of the expressions, and
the health state estimator estimates that the mental health state of the target person becomes worse when detecting that the positive expression score in a latest predetermined period has a decreasing tendency compared with an ordinary value or that the negative expression score in the latest predetermined period has an increasing tendency compared with the ordinary value as the feature associated with the temporal change of the expression.

7. The health care assisting device according to claim 1, wherein the expression recognizer calculates a score in which a degree of each of a plurality of kinds of the expressions is digitized from the image of the target person, and outputs the score of each expression as the expression recognition result,
the health state estimator selects or generates a positive expression score indicating the degree of the positive emotion or a negative expression score indicating the degree of the negative emotion from the scores of the plurality of kinds of the expressions, and
the health state estimator estimates that the mental health state of the target person improves when detecting that a fluctuation range of the positive expression score in a latest predetermined period has an increasing tendency compared with an ordinary value or that a fluctuation range of the negative expression score in the latest predetermined period has a decreasing tendency compared with the ordinary value as the feature associated with the temporal change of the expression.

8. The health care assisting device according to claim 1, wherein the expression recognizer calculates a score in which a degree of each of a plurality of kinds of the expressions is digitized from the image of the target person, and outputs the score of each expression as the expression recognition result,
the health state estimator selects or generates a positive expression score indicating the degree of the positive emotion or a negative expression score indicating the degree of the negative emotion from the scores of the plurality of kinds of the expressions, and
the health state estimator estimates that the mental health state of the target person becomes worse when detecting that an evening score tends to be relatively higher than a morning score in a daily fluctuation of the positive expression score or that the evening score tends to be relatively lower than the morning score in a daily fluctuation of the negative expression score as the feature associated with the temporal change of the expression.

9. The health care assisting device according to claim 1, wherein the health state estimator estimates that the mental health state of the target person becomes worse when detecting a change of an appearance ratio of a plurality of kinds of the expressions as the feature associated with the temporal change of the expression.

10. The health care assisting device according to claim 1, wherein the expression recognizer calculates a score in which a degree of each of the plurality of kinds of the expressions is digitized from the image of the target person, and outputs the score of each expression as the expression recognition result, and
the health state estimator estimates that the mental health state of the target person becomes worse when detecting that a difference between a score mean in a latest predetermined period and an ordinary value is larger than a threshold with respect to a part of or all the plurality of kinds of the expressions as the feature associated with the temporal change of the expression.

11. The health care assisting device according to claim 1, wherein the expression recognizer calculates a score in which a degree of each of the plurality of kinds of the expressions is digitized from the image of the target person, and outputs the score of each expression as the expression recognition result, and
the health state estimator estimates that the mental health state of the target person becomes worse when detecting that a fluctuation range of a score of a certain expression in a latest predetermined period has an increasing tendency compared with an ordinary value as the feature associated with the temporal change of the expression.

12. The health care assisting device according to claim 4, wherein the ordinary value is a value that is statistically obtained from the time-series data of the target person, the time-series data being stored in the storage.

13. The health care assisting device according to claim 4, wherein the latest predetermined period is a period longer than one day.

14. The health care assisting device according to claim 1, wherein the health state estimator estimates the mental health state using the time-series data of the target person for a plurality of days.

15. A health care assisting method for assisting mental health care of a target person using a computer, the health care assisting method comprising the steps of:
acquiring a plurality of images in which the target person is photographed in time series;
recognizing a feature of an expression of the target person from the acquired plurality of images;
storing expression recognition results of the plurality of images in a storage as time-series data;
detecting a feature associated with a temporal change of the expression of the target person from the time-series data stored in the storage, and estimating a mental health state of the target person based on the detected feature; and
outputting information on the mental health state of the target person based on the estimation result.

16. A non-transitory computer-readable recording medium storing a program causing a computer to perform operations comprising each of the steps according to claim 15.
